(19) United States
(12) Patent Application Publication
(10) Pub. No.: US 2019/0209364 A1
(43) Pub. Date: Jul. 11, 2019

(54) FEMALE CONDOM

(71) Applicant: Likai Jin, Fremont, CA (US)

(72) Inventor: Likai Jin, Fremont, CA (US)

(21) Appl. No.: 15/866,355

(22) Filed: Jan. 9, 2018

(51) Int. Cl. A61F 6/06

(52) U.S. Cl. CPC A61F 6/065

(57) ABSTRACT

A female condom includes a base section composed of elastic silicon that tends to hold shape. The base section is sized to remain on the outside of a vaginal cavity. The base section includes a hole. A tubular section is attached to the base section. The tubular section is designed to fit within the vaginal cavity. The tubular section is situated on the base section to entry through the hole in the base section to the tubular section. The tubular section is composed of medical grade silicon. The tubular section includes a tube infrastructure. The tube infrastructure is composed of silicon that is flexible but is of sufficient elasticity and resilience that the tube infrastructure tends to hold the tubular section of the female condom in an expanded state.

### BACKGROUND

Female condoms provide protection from pregnancy and sexually transmitted diseases. Female condoms are often made from latex.

### BRIEF DESCRIPTION OF THE DRAWINGS

FIG. 1 shows a simplified drawing of a female condom in accordance with an implementation.

FIG. 2 illustrates use of soft gel like silicon dots on the inside of a female condom in accordance with an implementation.

FIG. 3 shows a simplified top view of a female condom in accordance with an implementation.

FIG. 4 shows a simplified bottom view of a female condom in accordance with an implementation.

FIG. 5 illustrates compression by twisting of a female condom in accordance with an implementation.

### DESCRIPTION OF THE EMBODIMENTS

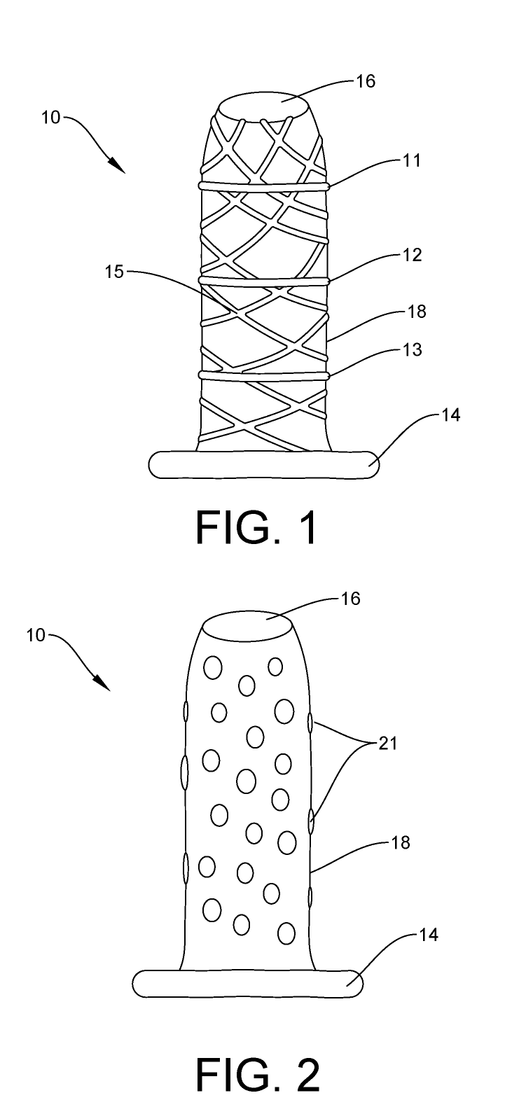

FIG. 1 shows a female condom 10 composed of various types of silicon. The outer area of a tubular section 18 of female condom 10 is made of a soft pliable medical grade silicon. Tubular section 18 of female condom 10 is reinforced by a tube infrastructure composed of silicon that is flexible but is of sufficient elasticity and resilience that the tube infrastructure tends to hold tubular section 18 of female condom 10 in an expanded state. The construction of female condom provides protection from pregnancy and sexually transmitted diseases can also offer greater protection of the vaginal area, particularly when there is damage to the vaginal wall or thinning, for example, as often happens with aging.

The tube infrastructure is composed of one or more rings of material, illustrated in FIG. 1 by a ring 11, a ring 12 and a ring 13. The use of three rings is illustrative as fewer or more rings may be present. In addition to the rings, additional structural material may be included. This is illustrated in FIG. 1 by a crisscrossing network 15 of structural elements embedded within or without tubular section 18 of female condom 10. For example, the tube infrastructure is located on the external surface of tubular section 18 of female condom 10, on the internal surface of tubular section 18 of female condom 10, embedded within the soft pliable medical grade silicon of tubular section 18 of female condom 10, or some combination of these. For example, tubular section 10 is reversible so that the tube infrastructure can be located by the user of female condom 10 on an inside of tubular section 10 or an outside of the tubular section 10.

A base section 14 is made of, for example, an elastic silicon that tends to hold its shape. A concave tip area 16 is impervious to sperm and disease causing bacterial and virus material. Concave tip area 16 has a concave shape to better fit the shape of the uterus at the intersection where the vagina meets the uterus. For example, concave tip area 16 has a depth of about one-half centimeter.

FIG. 2 illustrates that on an inside surface of tubular section 18 of female condom 10, raised dots 21 can be included that can add sensory tactile feedback when female condom 10 is used for its intended purpose. For example, raised dots 21 are soft gel-like silicon dots. Alternatively, soft gel-like silicon dots 21 can be embedded within tubular section 18 of female condom 10 or located on an external surface of tubular section 18 of female condom 10. For example, tubular section 10 is reversible so that the raised dots can be located by the user of female condom 10 on an inside of tubular section 10 or an outside of the tubular section 10.

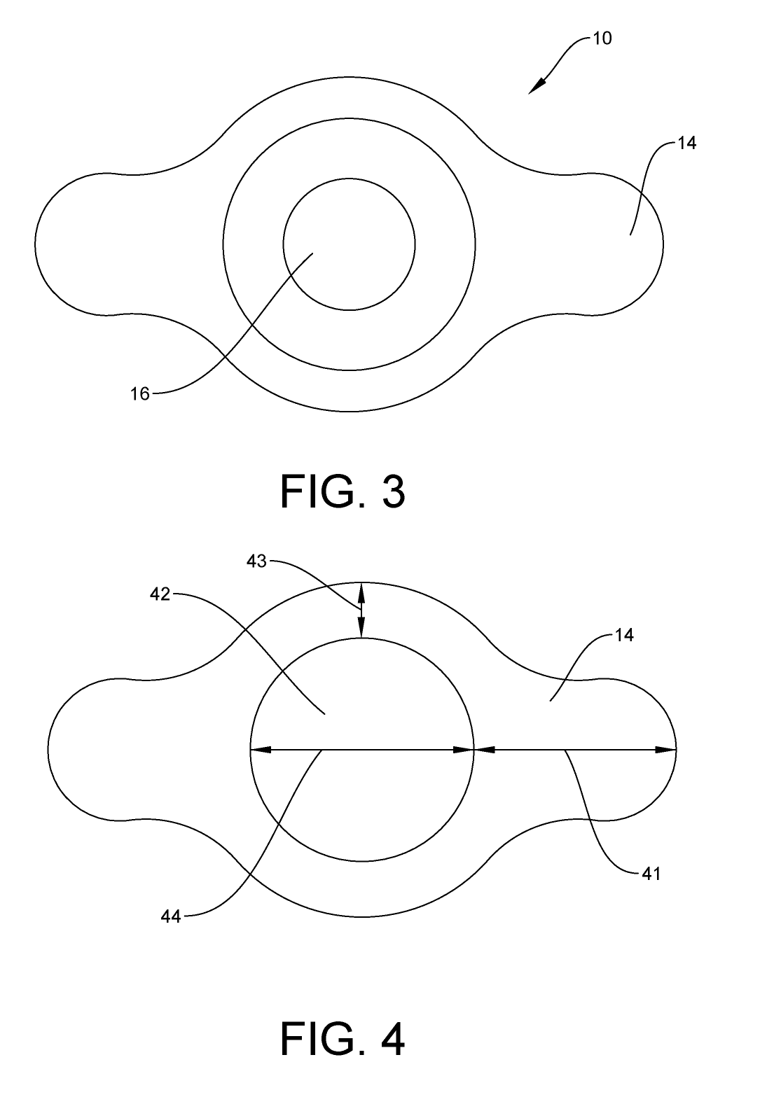

FIG. 3 shows a top view of female condom 10. Because of the tube infrastructure within tubular section 18 of female condom 10, the tube infrastructure tends to hold tubular section 18 of female condom 10 in an expanded state, as shown in FIG. 3.

FIG. 4 shows a bottom view of female condom 10. A hole 42 has a diameter 44 of, for example, between approximately two and four centimeters. A distance 41 is, for example, approximately two to three centimeters. A distance 43 is, for example 0.75 centimeters. The difference between distance 41 and distance 43 results in base section 14 having a width that is shorter than a length of base section 14. These distances can vary to accommodate differences in human anatomy. In general, diameter 44 of hole 42 and distance between base section 14 of female condom 10 and concave tip area 16 is sized approximately six to thirteen centimeters.

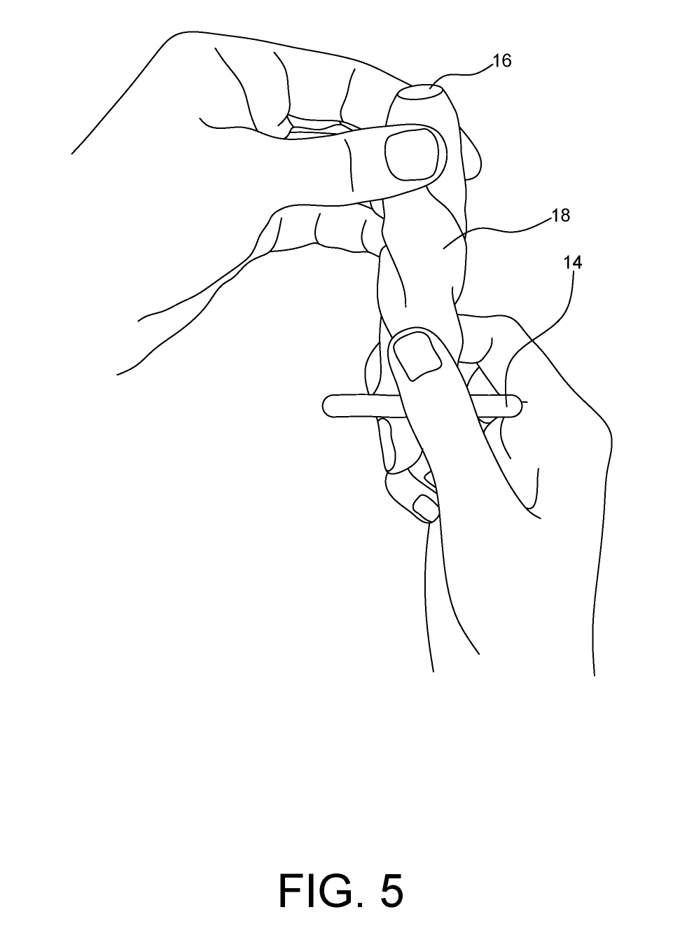

FIG. 5 illustrates twisting or folding of female condom 10 for the purpose of insertion into a vagina before use. As illustrated by FIG. 5, female condom 10 is twisted or folded to reduce a diameter allowing easy insertion. After twisting or folding female condom 10, a user can relax vaginal muscles and insert female condom 10, upwards and toward the user's tailbone until only base section 14 remains outside the vaginal cavity. A finger through opening in hole 42 can be used to gently help position female condom 10. Then base section 14 can be gently rotated or moved from side to side to allow the tube infrastructure of female condom 10 to revert to an expanded state within the vaginal cavity. For example, after twisting female condom 10 and inserting tubular section 18 into the vaginal area, base section 14 can be used to untwist and expand tubular section 18. A finger inserted through base section 14 into tubular section 18 can be used to check for correct installation.

Removal of female condom 10 can also be accomplished by base section 14 being gently rotated or moved from side to side while sliding female condom 10 out of the vaginal cavity.

The foregoing discussion discloses and describes merely exemplary methods and implementations. As will be understood by those familiar with the art, the disclosed subject matter may be embodied in other specific forms without departing from the spirit or characteristics thereof. Accordingly, the present disclosure is intended to be illustrative, but not limiting, of the scope, which is set forth in the following claims.

What is claimed is:

1. A female condom, comprising:
a base section composed of elastic silicon that tends to hold shape, the base section being sized to remain on the outside of a vaginal cavity, the base section including a hole; and,
a tubular section attached to the base section, the tubular section being designed to fit within the vaginal cavity, the tubular section being sized and situated on the base section to allow entry through the hole in the base section into the tubular section, the tubular section composed of medical grade silicon, the tubular section including:
a tube infrastructure, the tube infrastructure being composed of silicon that is flexible but is of sufficient elasticity and resilience that the tube infrastructure tends to hold the tubular section of the female condom in an expanded state.

2. A female condom as in claim 1 wherein the tube infrastructure includes a plurality of rings spaced along the tubular section.

3. A female condom as in claim 1 wherein the base section has a width that is shorter than a length of the base section.

4. A female condom as in claim 1 wherein the tube infrastructure includes a plurality of rings spaced along the tubular section and includes a crisscrossing network of structural elements embedded within or without the tubular section.

5. A female condom as in claim 1 wherein the tubular section has a concave tip on an end of the tubular section not connected to the base section.

6. A female condom as in claim 1 wherein the tubular section is reversible so that the tube infrastructure can be located on an inside of the tubular section or an outside of the tubular section.

7. A female condom as in claim 1 wherein either an inside or an outside of the tubular section is textured with raised dots.

8. A female condom as in claim 7 wherein the tubular section is reversible so that the raised dots can be located on an inside of the tubular section or an outside of the tubular section.

9. A method for inserting a female condom, comprising:
twisting or folding a tubular section of the female condom, the tubular section being designed to fit within a vaginal cavity and being sized and situated to allow entry into the tubular section through a hole in the base section, the tubular section composed of medical grade silicon;
inserting the twisted or folded tubular section into the vaginal cavity up to a base section of the female condom where the base section is composed of elastic silicon that tends to hold shape, the base section being sized to remain on the outside of a vaginal cavity and the base section including a hole that allows entry through the base section into the tubular section;
moving the base section back and forth or in a circular motion to allow a tube infrastructure within or without the tubular section to expand the tubular section within the vaginal cavity, the tube infrastructure being composed of silicon that is flexible but is of sufficient elasticity and resilience that the tube infrastructure tends to hold the tubular section of the female condom in an expanded state.

10. A method as in claim 9 wherein using a plurality of rings within the tube infrastructure to hold the tubular section of the female condom in an expanded state.

11. A method as in claim 9 wherein using a plurality of rings and a crisscrossing network of structural elements embedded within or without the tubular section to hold the tubular section of the female condom in an expanded state.

12. A female condom, comprising:
a base section composed of elastic silicon that tends to hold shape, the base section being sized to remain on the outside of a vaginal cavity, the base section including a hole; and,
a tubular section attached to the base section, the tubular section being designed to fit within the vaginal cavity, the tubular section being sized and situated on the base section to allow entry through the hole in the base section into the tubular section, the tubular section composed of medical grade silicon, the tubular section including:
a tube infrastructure, the tube infrastructure of sufficient elasticity and resilience that the tube infrastructure tends to hold the tubular section of the female condom in an expanded state, and
a concave tip on an end of the tubular section not connected to the base section.

13. A female condom as in claim 12 wherein the tube infrastructure includes a plurality of rings spaced along the tubular section.

14. A female condom as in claim 12 wherein the base section has a width that is shorter than a length of the base section.

15. A female condom as in claim 12 wherein the tube infrastructure includes a plurality of rings spaced along the tubular section and includes a crisscrossing network of structural elements embedded within or without the tubular section.

16. A female condom as in claim 12 wherein the tubular section is reversible so that the tube infrastructure can be located on an inside of the tubular section or an outside of the tubular section.

17. A female condom as in claim 12 wherein either an inside or an outside of the tubular section is textured with raised dots.

18. A female condom as in claim 17 wherein the tubular section is reversible so that the raised dots can be located on an inside of the tubular section or an outside of the tubular section.
